(19) United States
(12) Plant Patent Application Publication
(10) Pub. No.: US 2007/0061933 P1
(43) Pub. Date: Mar. 15, 2007

(54) Chrysanthemum plant named 'Cappuccino'

(76) Inventor: Keith G. Lintott, Bognor Regis (GB)

Correspondence Address: WINSTON & STRAWN LLP;PATENT DEPARTMENT, 1700 K STREET, N.W., WASHINGTON, DC, 20006, US

(21) Appl. No.: 11/513,578

(22) Filed: Aug. 31, 2006

Related U.S. Application Data
(60) Provisional application No. 60/715,014, filed on Sep. 8, 2005.

(51) Int. Cl. A01H 5/00

(52) U.S. Cl. PLT/287

(57) ABSTRACT

A new variety of Chrysanthemum plant named ‘Cappuccino’, having a slightly domed capitulum of white petalled, yellow/red cushion anemone type flowers and an upright, medium tall growth habit.

This application claims the benefit of U.S. Provisional Application No. 60/715,014, filed Sep. 8, 2005.

### LATIN NAME OF THE GENUS AND SPECIES

Botanical classification: Chrysanthemum morifolium.

### VARIETY DENOMINATION

The new Chrysanthemum variety denomination is ‘Cappuccino’.

### BACKGROUND OF THE INVENTION

The present invention comprises a new and distinct cultivar of Chrysanthemum botanically known as Chrysanthemum morifolium, and referred to by the cultivar name ‘Cappuccino’.

‘Cappuccino’, identified as 14063-0 originated from a cross made in a controlled breeding program in Chichester, West Sussex, United Kingdom. The female parent of the new variety is the unpatented variety ‘Cambridge’ and the male parent is the unpatented variety known as ‘Le Mans’. The new variety ‘Cappuccino’ has been asexually reproduced by vegetative cuttings, in Chichester, West Sussex, United Kingdom and the distinguishing characteristics are retained through successive generations of asexual reproduction.

### BRIEF SUMMARY OF THE INVENTION

‘Cappuccino’ is a cut type of Chrysanthemum plant variety having a flat capitulum form and white petal yellow/red cushion anemone type flowers.

### Comparison with Female Parent

Plants of the new Chrysanthemum variety ‘Cappuccino’ are dissimilar to the female parent ‘Cambridge’ in plant habit and growth rate. In side-by-side comparisons in Chichester, West Sussex, United Kingdom, under commercial practice, plants of the new Chrysanthemum variety ‘Cappuccino’ differed from plants of the female parent ‘Cambridge’ in the following characteristics:
1. The new variety ‘Cappuccino’ produces white petalled, yellow/red cushion anemone type flowers whereas the female parent ‘Cambridge’ produces orange anemone type flowers.
2. Plants of the new variety ‘Cappuccino’ have smaller and taller inflorescence than plants of the female parent ‘Cambridge’.

### Comparison with Male Parent

Plants of the new Chrysanthemum variety ‘Cappuccino’ are dissimilar to the male parent ‘Le Mans’ in plant habit and growth rate. In side-by-side comparisons in Chichester, West Sussex, United Kingdom, under commercial practice, plants of the new Chrysanthemum variety ‘Cappuccino’ differed from plants of the male parent ‘Le Mans’ in the following characteristics:
1. The new variety ‘Cappuccino’ produces white petalled yellow/red cushioned anemone type flowers whereas the male parent ‘Le Mans’ produces pink-white anemone type flowers.
2. Plants of the new variety ‘Cappuccino’ have larger and taller inflorescence than plants of the male parent ‘Le Mans’.
3. Plants of the new variety ‘Cappuccino’ have a 55 Short Day response whereas plants of the male parent ‘Le Mans’ have a 48 Short Day response.

### Comparison with the Chrysanthemum Variety ‘Apennine’

Plants of the new Chrysanthemum variety ‘Cappuccino’ are dissimilar to plants of the patent pending variety ‘Apennine’ (U.S. Plant patent application Ser. No. 11/396,882) in plant habit and growth rate. In side-by-side comparisons in Chichester, West Sussex, United Kingdom, under commercial practice, plants of the new Chrysanthemum variety ‘Cappuccino’ differed from plants of ‘Apennine’ in the following characteristics:
1. The new variety ‘Cappuccino’ produces white petalled yellow/red cushioned anemone type flowers whereas ‘Apennine’ produces white petalled green cushioned anemone type flowers.
2. Plants of the new variety ‘Cappuccino’ have smaller and taller inflorescence than plants of ‘Apennine’.
3. Plants of the new variety ‘Cappuccino’ have lighter and smaller foliage than plants of ‘Apennine’.

### BRIEF DESCRIPTION OF ILLUSTRATIONS

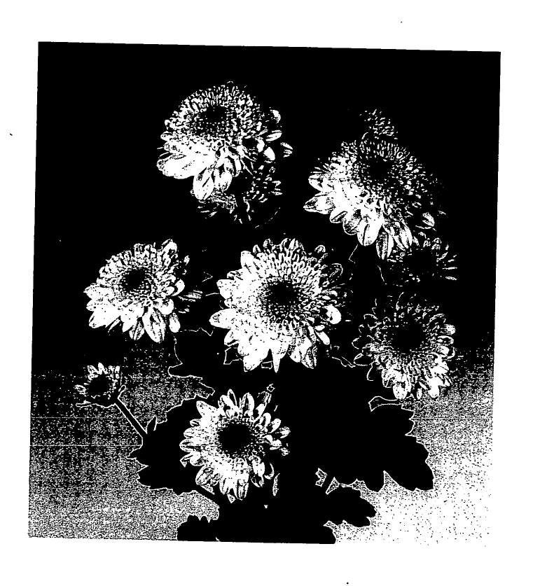

Typical specimens of the plant and flowers for the new Chrysanthemum variety ‘Cappuccino’ are shown in the accompanying illustration printed from a digital photograph. The colors shown are as true as possible within the usual limits of this kind of illustration. FIG. 1 is a closer view, showing inflorescence and foliage characteristics of the new cultivar in greater detail. The plant shown in the illustration is 55 days from the onset of Short Days.

### DETAILED BOTANICAL DESCRIPTION

The following description of the new Chrysanthemum variety ‘Cappuccino’ is of plants grown for 55 Short Days in a greenhouse in Chichester, West Sussex, United Kingdom, in the month of February. The cultivar has not been observed under all possible environmental conditions. The phenotype may vary significantly with variations in the environment such as temperature, length of day and light intensity, without any variance in genotype. The commercial classification of the new variety is a cut Chrysanthemum.

Plants of the new variety have been grown successfully under temperature conditions averaging about 18° C. at night and about 18° C. to 25° C. during the day under light conditions of about 5,000 to 6,000 foot candles. The plants respond well to the use of growth retardant, such as three B9 treatments at about 2500 ppm. To produce a commercial product, the plants have the center bud removed. It has been observed that the shelf life of the new variety is about 18 days with a response time of about 8 weeks. The new variety is suitable for growth in a temperature range of 18° C. to 25° C. The new variety may be produced as a spray Chrysanthemum. The following description is with respect to a plant produced as a spray Chrysanthemum. In the description of this new Chrysanthemum variety, color values have been taken from The Royal Horticultural Society Colour Chart (R.H.S.C.C.).
Plant:
   Plant type.—anemone type cut Chrysanthemum.
   Habit.—Upright.
   Height (cm).—90-110.
   Width (cm).—8-12.
   Stem.—Length (cm): 80-105 Diameter (cm): 0.6-0.8 Texture: Slightly rough, slightly pubescent Color: near 143A.
   Branching characteristics.—Lateral Branches: Length (cm): 5.5-8.0 Diameter (cm): 0.2-0.3 Texture: Slightly rough, slightly pubescent.
   Number of breaks from pinch.—9-16.
   Response time.—near 55 days.
   Vigor.—Medium/Tall.
   Shelf life.—near 18 days.
   Life of blooms.—near 18 days.
   Disease (susceptibility/resistance observed).—None observed to date.
   Pest (susceptibility/resistance observed).—None observed to date.
   Drought and temperature (susceptibility/resistance observed).—None observed to date.
   Growth retardant type and treatment.—3 applications of 2.5 gm/liter B9 @21, 25 and 28 days after sticking unrooted cuttings. The plants were grown for 2 weeks in Long Day conditions (20 hours of light) and then transferred to Short Day conditions (13 hours of dark).
   Propagation.—Type: Vegetative propagation via stem cuttings Time to rooting: 12 days with soil temperatures of 22° C. Rooting habit: After 7 Long Days, the first roots emerge and form root primordia. In 14 Long Days, a complete root system is developed.
Foliage:
   Number of leaves per lateral branch.—37-44.
   Compound or single.—Single.
   Arrangement of leaves.—Alternate. Shape of Leaf: Typically 5 lobed Size of Leaf: Width (cm): 4-6.5 Length (cm): 4-9.5 Leaf Apex: Rounded Base: Truncate Attachment: Petioled Aspect: Slightly undulating Margin: 5 lobed palmate Surface characteristics: Top: Slightly pubescent Bottom: Slightly pubescent.
   Petiole.—Color: near 138A Length (cm): 1-2 Diameter (cm): 2.2.5 Surface: Slightly pubescent, slightly rough.
   Venation.—Net, prominent lateral and mid-veins at underside Color: upper side: near 144B under side: near 144B.
   Color.—Mature Leaf, upper side: near 137A; underside: near 137C Young Leaf, upper side: near 137A; under side: near 137C.
Flower:
   Flower appearance.—Matte.
   Flower type.—Anemone.
   Flower form.—Flat.
   Flower shape.—Round.
   Flowering habit.—Cylindrical.
   Number of blossoms per branch.—9-16.
   Typical and observed flowering season.—Year Round.
   Inflorescence form.—Corymbiform Depth of fully expanded blossoms (cm): 1-2 Diameter of fully expanded blossoms (cm): 5-6 Anemone center diameter (cm): 2-2.5.
Phyllaries: Number: 21-23 Color: near 137C Length (cm): 0.6-0.8 Width (cm): 0.2-0.3 Texture/Appearance: Smooth, slightly pubescent.
   Pedicel.—Length (cm): 5.5-8.0 Diameter (cm): 0.2-0.3 Angle from stem (degrees): 15-20 Color: near 143A Surface: Slightly rough, slightly pubescent Habit: Slightly curving Strength: Strong.
   Ray florets.—Form/Shape: Straight with a few tubular Texture/Appearance: Smooth, slightly ridged Number per flower: 45-50 Length (cm): 2-2.5 Width (cm): 0.5-0.8 Apex: Round Base: Tubular Margin: Entire.
   Disc florets.—Form/Shape: Cylindrical Texture/Appearance: Smooth, dull Number per flower: 140-150 Length (cm): 0.8-1.0; Width (cm): <0.1 0.15 Diameter of disc (cm): 0.5-0.6.
   Fragrance.—None.
   Flower bud (at onset of color).—Length (cm): 0.6-0.8 Diameter (cm): 0.8-1.0 Form/Shape: Round, globular.
   General flower color.—1. Ray florets, upper side: Immature: near 155B Mature: near 155B Older/Fading: near 155B 2. Ray florets, under side: Immature: near 155C Mature: near 155C Older/Fading: near 155C 3. Ray florets, tube, outer side: near 158B 4. Ray florets, tube, inner side: near 158A 5. Disc florets: Immature: near 6C; tip near 42B Mature: near 6D Older/Fading: near 5D 6. Bud: near 137A.
Flower progression with age.—Flowers do not fade with age.
Reproductive organs:
   Gynoecium present on ray and/or disc florets.—
   Ray florets per individual flower.—Pistillate. Pistil number: 45-50 Stigma color: near 151A Stigma shape: Forked Style color: near 151A Style length (cm): 0.1-0.4.
   Disc florets per individual flower.—Pistillate Pistil number: 140-150 Stigma color: near 151A Stigma shape: Forked Style color: near 151A Style length (cm): 0.15-0.2.5.
   Androecium.—Not observed.
   Fruit and seeds.—None observed.

What is claimed is:

1. A new and distinct variety of Chrysanthemum plant, substantially as described and illustrated herein.
